(19) United States
(12) Patent Application Publication
(10) Pub. No.: US 2026/0306739 A1
(43) Pub. Date: Oct. 8, 2026

(54) ADJUSTABLE PRELOAD FOR SPRING-LOADED PUMPING ELEMENTS IN AN INFUSION PUMP

(71) Applicant: CareFusion 303, Inc., San Diego, CA (US)

(72) Inventors: Richard O'Connor, Castletroy (IE); Kevin O' Brien, Tipperary (IE); Vadym Sologub, Limerick (IE); Kevin O'Sullivan, Limerick (IE)

(21) Appl. No.: 19/171,087

(22) Filed: Apr. 4, 2025

(51) Int. Cl. A61M 5/145; A61M 5/142

(52) U.S. Cl. CPC A61M 5/145; A61M 2005/14208; A61M 2005/14506

(57) ABSTRACT

Disclosed is an infusion pump that includes spring-loaded pumping elements with an adjustable preload for preserving elasticity of a fluid tubing loaded within the pump. Each pumping element of the pumping mechanism is biased by a respective preloaded spring element positioned between a portion of the pumping element and a movable support reference, with the preloaded spring element contributing to a corresponding compression force on the fluid tubing. An actuator device causes the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.

### TECHNICAL FIELD

The present disclosure relates generally to intravenous infusion pump mechanisms. In particular, the present disclosure relates to operation of an infusion pump on an infusion set.

### BACKGROUND

In the medical field, infusion pumps are used often to administer accurate amounts of infusates from an external bag to a patient via an intravenous (IV) administration set. The IV administration set is used to administer fluids, medications, or blood products directly into the patient's bloodstream. It typically consists of a plastic or flexible tubing with connectors on either end—one end attaches to a fluid source such as a saline bag or medication vial, while the other end connects to a needle or catheter inserted into a vein. The IV administration set includes a pumping segment—a section of tubing in the IV administration set—that loads into the infusion pump and passes through the pump's rollers or squeeze mechanism. As the pump compresses and releases the tubing of the pumping segment in a rhythmic motion, it generates a pulsatile flow, effectively propelling fluid through the tubing of the IV administration set and into the patient's bloodstream.

### SUMMARY

As existing volumetric infusion pumps operate by compressing an elastic segment of the IV administration set against a platen to deliver fluid, the repeated mechanical compression causes the elastic segment to undergo localized plastic deformation. Initially, the elastic segment exhibits elastic behavior, allowing it to return to its original shape after each compression cycle. However, over time, the continuous stress and strain cause the material in the compressed regions to exceed its elastic limit, transitioning into plastic behavior. This plastic deformation results in permanent changes to the shape and thickness of the elastic segment, which affects its ability to maintain a consistently high compression force against the platen. Consequently, this transition from elastic to plastic behavior leads to a change in flow rate accuracy, causing the infusion pump to deliver fluid at a rate that may deviate from the intended or calibrated flow rate, thereby impacting the reliability and precision of fluid delivery over time.

Typically, the IV administration set is replaced approximately every 72 hours to maintain accurate fluid delivery and to minimize the risk of complications such as infection or mechanical failure due to material fatigue. Enhancing the material properties to resist plastic deformation and maintain consistent elasticity would not only improve flow rate accuracy over an extended period but also reduce the frequency of set changes, resulting in cost savings, reduced labor for healthcare providers, and less disruption for patients.

The present disclosure addresses the aforenoted problems by providing an infusion pump pumping mechanism with an adjustable preload for spring-loaded pumping elements. According to various implementations, the infusion pump, comprises: a pumping mechanism comprising a housing and at least one pumping element configured to periodically compress a fluid tubing filled with a fluid against a rigid support surface when the fluid tubing is received by the infusion pump to pump a fluid through the fluid tubing in a predetermined direction, a movable support reference configured to move with respect to the housing of the pumping mechanism; at least one preloaded spring element positioned between the movable support reference and a portion of the at least one pumping element, causing the at least one pumping element to exert a compression force on the fluid tubing thereby compressing the fluid tubing against the rigid support surface; and an actuator device configured to cause, while the at least one pumping element is periodically compressing the fluid tubing, the movable support reference to move towards or away from the housing and the portion of the at least one pumping element so as to increase or reduce the preload of the at least one preloaded spring element and the corresponding compression force exerted on the fluid tubing by the one or more pumping elements. Other aspects include corresponding systems and methods for implementation of the corresponding apparatus and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of an example infusion pump showing an intravenous (IV) infusion set loaded in place within the infusion pump, according to various aspects of the subject technology.

FIG. 2 depicts an example patient care unit shown, according to various aspects of the subject technology.

FIGS. 3A-3C depict example pumping mechanisms of a large volume infusion pump including a pumping mechanism having two occluder valves, according to various aspects of the subject technology.

FIGS. 4A-4C depict example pumping mechanisms that include spring-loaded pumping elements with an adjustable preload, according to various aspects of the subject technology.

FIG. 5 depicts an example process for operating an infusion pump that includes spring-loaded pumping elements with an adjustable preload for preserving elasticity of a fluid tubing loaded within the pump, according to aspects of the subject technology.

FIG. 6 is a conceptual diagram illustrating an example electronic system for operating an infusion pump that includes spring-loaded pumping elements with an adjustable preload for preserving elasticity of a fluid tubing loaded within the pump, according to aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Conventional pumping mechanisms within large volume infusion pumps have to work at a consistent maximum force on every cycle even if the pressure is not present within the set. Such systems are pre-calibrated to a tolerance that is generally encountered but not variable. As will be described further, the subject technology provides the ability to adjust a preload on spring-loaded pumping elements so that the occlusion force may be backed off in low pressure situations. As the pressure increases, an actuator can adjust the force to a worst case situation or step up the force as needed.

The subject technology includes an infusion pump having a pumping mechanism with an adjustable preload for spring-loaded pumping elements, such as occluders and/or pumping fingers. The occluders and/or fingers within the pumping mechanism are biased by a respective preloaded spring element positioned between a portion of the pumping element (e.g., the end opposite the end responsible for compressing the tubing) and a movable support reference. Each preloaded spring element contributes to a corresponding compression force provided by the pumping element on a fluid tubing loaded within the pump. An actuator device causes the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.

Some of the advantages of embodiments consistent with the present disclosure include the compression of an IV infusion set being variable and commensurate with pressure inside the infusion set. By appropriately adjusting the preload of the pumping elements, the segment's elastic material is not repeatedly subject to maximum forces that would otherwise be sufficient for all pressures, thereby reducing the degradation of the material, and thus reducing the corresponding degradation in the rate accuracy over time, in addition to a reduction in spallation. Elasticity of a pumping segment loaded within the infusion pump is preserved and the life of the pumping segment extended (e.g., extending the set change interval to over 72 hours).

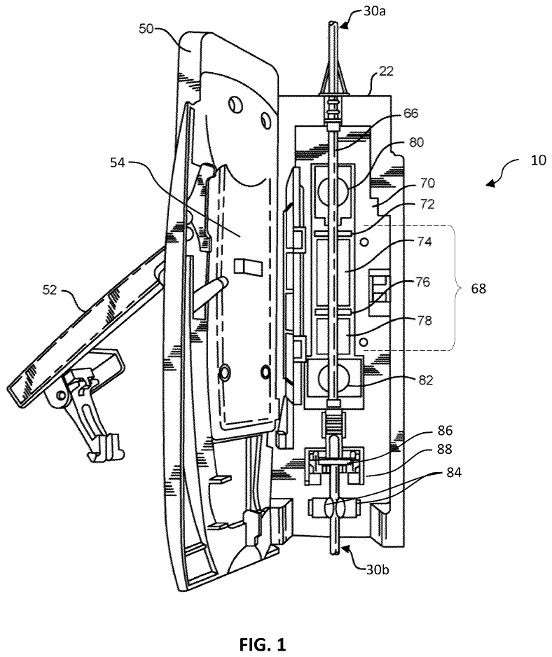

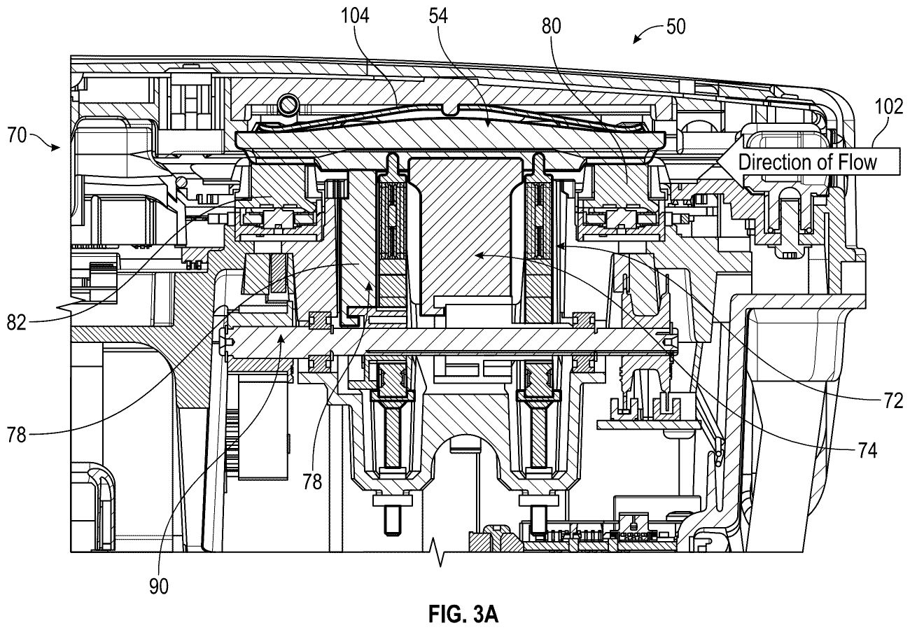

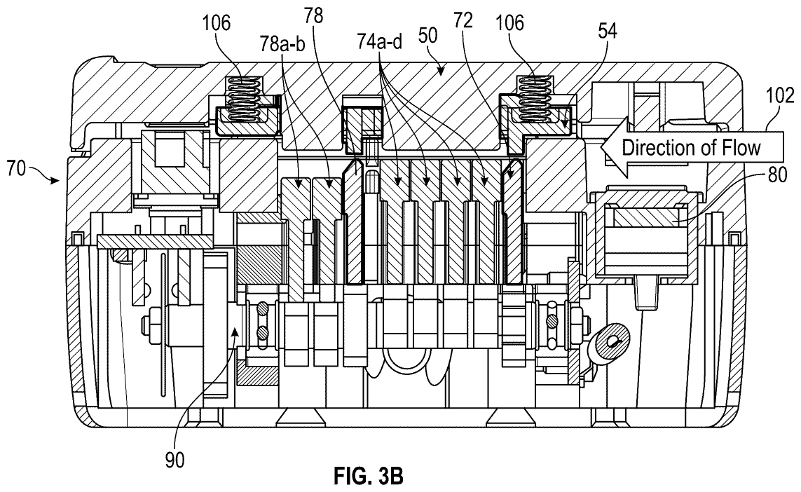

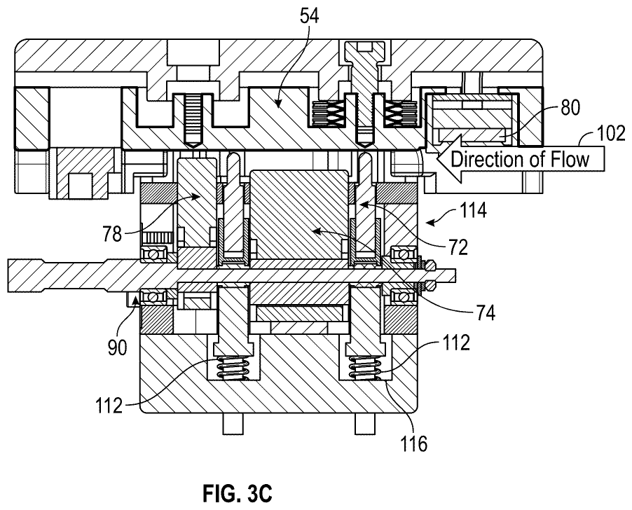

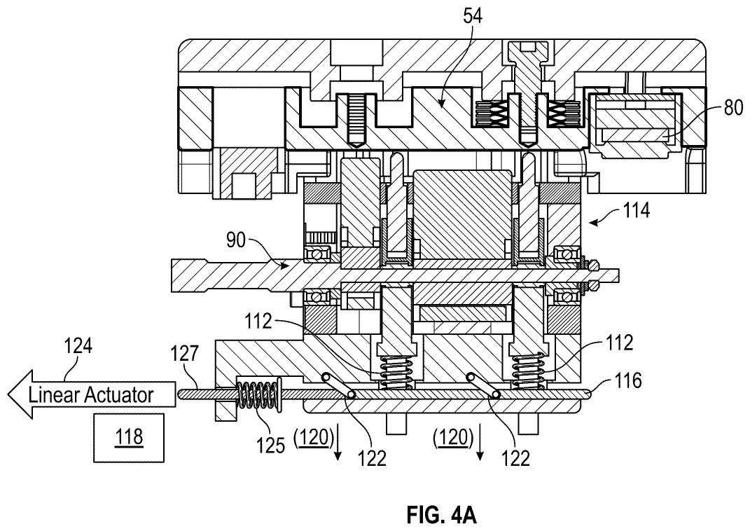

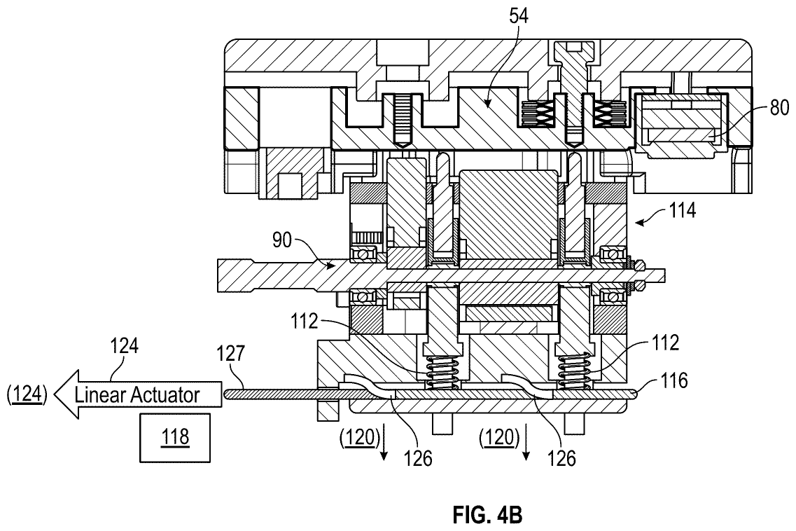

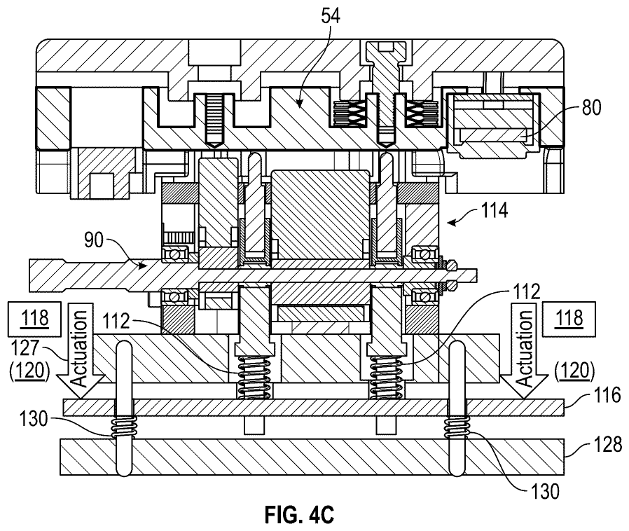

FIG. 1 depicts a perspective view of an example infusion pump showing an intravenous (IV) infusion set 30 loaded in place within the infusion pump, according to various aspects of the subject technology. An infusion system for parenteral infusion of a medical fluid to a patient comprises a pump unit 10, a major part of which comprises a housing or casing 22 which accommodates, in manner known per se, a cam (see FIGS. 3A-3C, 4A-4C) controlling a pumping mechanism including a plurality of pumping elements, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls.

As shown in FIG. 1, the infusion pump 10 (aka an “infusion device”) is shown in perspective view with the front door 50 open, showing the upstream portion 30a of the IV infusion set (e.g., portion between fluid container and the infusion pump 30) and downstream portion 30b (e.g., portion between the infusion pump 10 and a patient) in operative engagement with a pumping mechanism 70 of the infusion pump 10. The infusion set tubing may include a continuous fluid conduit formed from the upstream fluid line 30a to the downstream fluid line 30b, extending from a respective fluid supply to a patient, through which fluid is acted upon by the pump to move fluid downstream to the patient. The infusion pump 10 directly acts on a pumping segment 66 of the infusion set that passes through the pumping mechanism 70 of the pump. Specifically, the pumping mechanism 70 includes a plurality of pumping elements 68 and acts as a flow control device of the pump to move fluid though the conduit. The depicted references 30a-b, 66 may be used to describe herein portions of one continuous fluid line or, in some implementations, may individually describe portions that are fluidly connected together to form a continuous fluid line.

As shown in FIG. 1, the door or face plate 50 which may be opened to reveal the internal loading mechanism for an infusion set. Within the housing of the infusion pump (e.g., behind the door or face place), the infusion pump includes a pumping mechanism configured to operate on and compress an elongated compressible channel of the pumping segment 66, when loaded within the pumping mechanism of the pump. The pumping mechanism 70 includes a group of serially-aligned pumping elements 68 (e.g., occluders 72, 76 and/or pumping fingers 74, 78) configured to compress the elongated compressible channel (e.g., an IV tubing segment) against a rigid tubing support surface 54 when the pumping segment 66 is loaded within the pumping mechanism.

The infusion set includes an intermediate section of resiliently compressible tubing 66, for example of silicone rubber and, in some implementations, upper and/or lower fittings which each tubing section may be connected respectively with a respective upper line 30a and with the lower line 30b. In use, each upper line 30a extends upwardly to a source of the medical fluid to be administered whilst the lower line 30b extends from the infusion pump to an infusion needle or the like inserted into the patient. In use, the compressible tubing 66 (or “pumping segment”) is extended across the face or deck of the pump unit so that its fittings (not labeled) are received in respective brackets respectively and so that the tubing segment extends over a pumping mechanism. In the depicted example, the pumping mechanism 70 includes a four element pump assembly 72, 74, 76, 78. (See also FIG. 3A.) In the depicted example, the infusion set is fitted in place in this fashion whilst the door 50 is in the open position. After the infusion line has been so fitted, the door 50 may be moved to the closed position and is secured by a catch 52 which may include a lever mounted on the outer edge of the door. When the door is closed, the rigid support surface 54 is locked below the pump assembly so that the pumping segment 66 may be compressed against the surface 54 by elements 72-78.

The depicted pumping mechanism 70 includes respective elements 72, 74, 76, 78 (e.g., plungers, fingers, and/or occluders) that are moveable by a cam system inwards and outwards from the face or deck of the pump to compress a respective tubing segment against a platen 54 (e.g., a counter surface or anvil) to propel fluid within the infusion line. In order to make it easier to maintain sterile conditions, these pumping elements may be covered by a thin flexible membrane, (not shown), sealed at its edges with respect to the deck. The pumping elements of the pump assembly periodically press the flexible resilient tubing against the platen 54 which, as described previously, may be configured on an opposite side, for example, on an inner portion of the door 50.

The type of pumping mechanism may vary, including in the number of pumping elements (e.g., pumping fingers. In the depicted example, the pumping mechanism 70 includes an upstream occluding element or occluder 72, a primary pumping element or pump finger 74, a downstream occluding element occluder 76, and a secondary pumping element or flow finger 78. The pumping mechanism (and mechanisms used in other linear peristaltic pumps) operate by sequentially pressing on a segment of the fluid conduit by means of the cam-following pumping elements (e.g., occluders, pumping fingers and flow fingers) 72, 74, 76, and 78, which in the depicted example make a four finger pump assembly. Each element may be sequentially activated by a respective cam lobe on a camshaft to apply a downward compression against the compressible tubing 66 (or pumping segment), to move the fluid in the tubing 66 downstream. Pump finger 74 may include multiple intermediate elements or fingers (see FIG. 3B) that sequentially activate according to positioning of the cam lobes. In some implementations, the pressure is applied in sequential locations of the conduit, beginning at the upstream end of the pumping mechanism, and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

An upstream pressure sensor 80 may also be included in the pump 10. The upstream pressure sensor may be mounted to pumping mechanism 70 or located adjacent and upstream in relation to the pumping mechanism 70 between a fluid supply and the pumping mechanism 70, so that the connection of the correct fluid supply with the correct pump may be verified before any fluid is pumped to the patient. A downstream pressure sensor 82 is also included in the example infusion pump 10 at a downstream location with respect to the pumping mechanism, that is, at a location between the patient and the flow control device, so that the connection of the correct fluid supply with the correct pump may be verified before any fluid is pumped to the patient. Downstream pressure sensor 82 may be used to detect a pressure change adjacent to occluder 76 and/or downstream finger 78, to determine whether these elements are functioning properly.

The infusion pump 10 may also include an air-in-line (AIL) sensor 84. In some implementations, air-in-line sensor 84 may be mounted at a bottom end of chassis 22. air-in-line sensor 84 may include two sensing elements disposed on opposite sides of a slot through which the infusion tubing 66 passes. The air-in-line sensor 84 includes a signal (e.g., an ultrasound signal) traveling across the tubing that includes the infusion fluid). Accordingly, infusion tubing 66 should be seated appropriately so that the ultrasound signal in air-in-line sensor 84 traverses the fluid.

In some implementations, the pumping segment 66 may include a cartridge 86 that clips into a compartment 88 of the pump for proper positioning. In the depicted example, the cartridge 86 includes a flow stop that is received into a corresponding compartment 88 in the casing 22 above the air-in-line sensor 84. In this regard, the flow stop cartridge plugs into the compartment and facilitates alignment of the pumping segment 66 with the pumping components, and further facilitates alignment of the downstream line 31 with the air-in-line sensor 84.

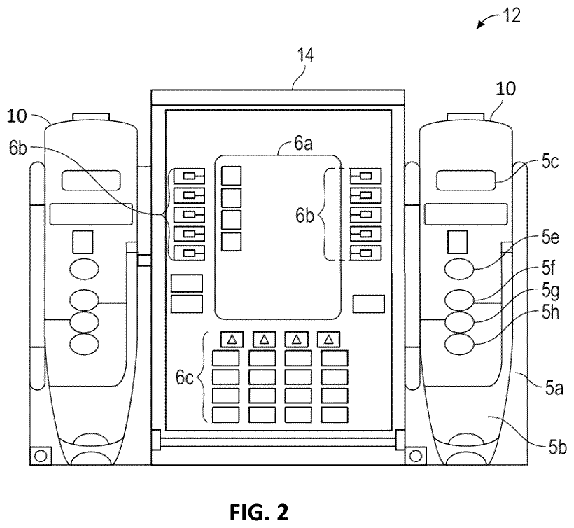

FIG. 2 depicts an example patient care unit 12 shown, according to various aspects of the subject technology. FIG. 2 shows two functional infusion pumps 10 (e.g., “infusion pump modules”) mounted at either side of a main frame infusion controller 14, and the displays and control keys of each, with the main frame infusion controller 14 being capable of programming both infusion pumps. The infusion pump includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 10. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 10, such as alert indications (e.g., alarm messages). Control keys 5e-h may exist for programming and controlling operations of the infusion pump as desired. In some implementations, the control keys may be presented as interactive elements on the display 5c (e.g., touchscreen display). The main frame and/or functional module may also include audio alert equipment in the form of a speaker (not shown).

The main frame infusion controller 14 of the patient care unit 12 includes a display 6a for visually communicating various information, such as the operating parameters of a connected pump and alert indications and alert messages, and control keys 6b and 6c for selecting and/or setting control parameters and/or options for controlling the patient care unit 12 and connected modules. The main frame infusion controller 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The main frame infusion controller 14 may include a communications system (not shown) with which the main frame infusion controller 14 may communicate with external equipment such as a medical facility server or other computer and with a portable processor, such as a handheld communication device or a laptop-type of computer, or other information device that a clinician may have to transfer information as well as to download drug libraries to a functional module 10. The communication module may be used to transfer access and interaction information for clinicians encountering the main frame infusion controller or device coupled therewith (e.g., pump 10 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 10, such as in cases where a main frame infusion controller is not used, or in addition to one with the main frame infusion controller 14. Further, information input devices need not be hard-wired to medical instruments, information may be transferred through a wireless connection as well. Additionally, other types of modules may be connected to the pump modules or to the main frame infusion controller such as a syringe pump module, patient controlled analgesic module, end tidal CO₂ monitoring module, oximeter monitoring module, or the like.

FIGS. 3A-3C depict example pumping mechanisms 70 of a large volume infusion pump including a pumping mechanism having two occluder valves 72, 76, according to various aspects of the subject technology. While a large volume infusion pump is depicted and used to describe the subject technology, the subject technology may be applied to numerous other pumps that utilize compression of tubing to effect the pumping action.

Infusion devices, such as those that include large volume infusion pumps (LVPs), may use a linear peristaltic or similar type of mechanism that creates the pumping action by way of squeezing the IV tubing to generate the pressure that causes the IV medication to flow through the IV tubing and ultimately into the patient. The squeezing action can also be used as a mechanism to close off the flow. This is used in pumping mechanisms that operate in a cyclical fashion by operating in a cycle that includes a filling portion and a delivery portion.

A typical medical pump for IV infusion delivery uses valving that includes an intake (or fill) occluder valve 72, and an outflow (or delivery) valve 76. The pumping mechanism 70 may include a single pump finger 74 in between occluders 72, 76, as depicted in FIGS. 1 and 3A, or may include multiple pump fingers 74a-d, as depicted in FIG. 3B. The occluders and plunger/fingers coordinate with each other in programmable, sequential steps, controlled by a cam system 90 to have two phases: 1) a filling phase, and 2) a delivery phase. The occluders and fingers cooperate to compress the pumping segment 66 of the IV infusion set against a rigid platen or support surface 54 to move fluid within the pumping segment 66 (and tubing 30) by sequentially compressing the tubing, thereby causing a flow in a direction 102 according to the particular compression sequence of the occluders.

Proper operation of the pumping mechanism requires complete closure of the occluder valves 72, 76 when they are not expected to be open. Incomplete closure will produce leakage, which can produce uncontrolled flow resulting in either, not enough medication, or too much medication being delivered to the patient.

These sequences can repeat through multiple cycles. To specify, when the pump finger 74 (e.g., of FIGS. 1 and 3A) is lifted from the tubing segment during the filling phase, there may be a disruption in the continuous infusion process.

Fluid is moved through the IV line 30, 66 using the coordinated sequence of actions involving an upstream occluder 72, a pump finger 74, a downstream occluder 76, and a flow finger 78. During the medication infusion process, in the filling phase, the upstream occluder 72 lifts to suck the medication into the tubing segment, which creates a pause, followed by the delivery phase to push the fluid out. In this regard, the upstream occluder 72 compresses the IV line to prevent backflow toward the pumping segment 66, while the downstream occluder 76 remains open, allowing forward flow. The pump finger 74 then compresses the tubing at a specific point, pushing a precise amount of fluid downstream. As the pump finger 74 completes its stroke, the downstream occluder 76 closes to prevent fluid from flowing backward, and the upstream occluder 72 may release to allow fluid to refill the portion of the pumping segment 66 behind pump finger 74.

The flow finger 78 may fine tune the rate of fluid delivery by partially compressing the pumping segment 66 to control the flow as needed. This process operates in a repetitive, tightly synchronized cycle to ensure accurate and consistent fluid delivery. In coordinating the occluders and pump fingers, the pumping mechanism 70 ensures unidirectional flow 102.

In the depicted examples, the platen 54 is integrated into the door 50 of the infusion pump 10. However, the platen 54 may integrated into the pumping mechanism 70 in other implementations (e.g., when the pumping segment 66 loaded from a side of the mechanism). The platen 54 provides a rigid, flat surface that provides support for the tubing, so that a consistent pressure can be applied to the tubing by the pumping occluders and fingers to ensure precise fluid delivery.

According to various implementations, the platen 54 may be spring loaded and/or supported by one or more spring mechanisms. In this manner, the force provided by the spring(s) may counter the occluding force and facilitate an even distribution of pressure along the tubing. In some implementations, as depicted in FIG. 3A, the platen 54 may be supported by one or more leaf springs 104 (e.g., flexible steel or metal alloy). In some implementations, as depicted by FIG. 3B, the platen may be supported by one or more coiled compression springs 106. In this regard, the compression force applied by the occluders and/or fingers may, at least in part, be a function of the force or resistance provided by the spring loaded platen. In some implementations, the amount of preload on the spring elements supporting the platen may be precalibrated, for example, based on the type of administration set materials expected to be used by the pump 10, and the amount of pressure in the set at any given point in time.

Additionally or in the alternative, as depicted in FIGS. 3A and 3C, each of the depicted occluders 72, 76 may be biased with a respective compression spring 112 which causes the occluder to exert a default compression force on the compressible tubing 66 loaded within the pump 10 and positioned between the platen 54 and the occluders 72, 76. In such implementations, each compression spring 112 places the occluder and/or finger in a compression state by default, while the cam 90 rotates to lift each occluder and/or finger from its default compression state according to a predetermined pumping cycle. In FIG. 3B, the cam motion directly causes the compression state, while the spring-loaded platen 54 provides calibration of the force applied to the tubing therebetween.

In some implementations, a compression force or resistance component of the spring(s) supporting the platen may be adjusted by a mechanical actuator to adjust the occluding force applied to the pumping segment 66. In this regard, the platen 54 may act as a reference surface for the occluders and/or fingers of the pumping mechanism 70, by which a force against the occluders/fingers can be increased to increase the overall compression of the tubing, or reduced to reduce the overall compression of the tubing. In some implementations, a mechanical actuator may adjust the position of the door latch or the hinge (or both) to adjust the overall compression force.

FIGS. 4A-4C depict example pumping mechanisms that include spring-loaded pumping elements with an adjustable preload, according to various aspects of the subject technology. In the depicted examples, the disclosed pumping mechanism 70 includes a housing 114 within which a fluid tubing 66 may be loaded and pumping elements 72, 26 are configured to periodically compress the fluid tubing 66, while loaded, to pump the fluid through the tubing. While the pumping elements shown and described relate to occluders 72, 76, it is understood that the described mechanism may apply to any pumping elements described herein, including fingers 74, 78.

According to various implementations, a respective compression spring 112 is positioned between a support reference 116 and each occluder 72, 74, causing each occluder to exert a default compression force on the fluid tubing within the housing 114. In such implementations, the support reference 116 is movable, for example by an actuator 118, such that it's position within the housing 114 may be adjusted. In this manner, the support reference 116 acts as a reference surface for the sprung elements, and an adjustment to its position changes a pre-load compression on the compression springs 112.

In some implementations, the actuator device 118 includes a motor controlled mechanism that directly acts on the support reference 116 to adjust it's location relative to the tubing loaded within the pump (e.g., vertical position relative to a horizontally positioned tubing). In some implementations, the actuator device includes a piezoelectric actuator. In some implementations, the actuator device includes a hydraulic actuator. The actuator 118 is configured to, while the pumping elements 68 of the pumping mechanism (e.g., occluders and/or fingers) are periodically compressing the fluid tubing 66, cause the support reference 116 to move away (120) in an opposite direction from the tubing so as to reduce the preload on the compression springs and thus reduce compression force exerted by the pumping elements 68 on the fluid tubing.

In some implementations, a processor of the infusion pump may monitor a pressure of the fluid within the IV infusion set and/or compressible tubing 30, 66 via a pressure sensor 80 along the flow path. In this regard, the processor may be configured to activate the pumping elements 68 to compress the fluid tubing and, determine, using the pressure sensor(s), while the pumping elements 68 are periodically compressing the pumping segment 66 loaded in the pump 10, that the pressure of the fluid within the tubing satisfies a pressure threshold for a predetermined period of time. When the pressure satisfies the pressure threshold for the requisite time period, the actuator 118 may be activated to cause the support reference 116 to move in a direction (120) so as to reduce tension/preload in the compression springs 112 and thus reduce the compression force exerted by the pumping elements 68 on the pumping segment 66. When the pressure measured by the pressure sensor 80 satisfies a second threshold, the actuator may be activated to cause the support reference to move to a default position so as to restore a default compression force exerted on the fluid tubing. In this regard, the second threshold may be indicative of a failure of the actuation system, such that the system would disengage and default to a position of maximum pre-load on the sprung element(s).

While three exemplary configurations of the support reference 116 and associated actuator 118 are described herein, it is understood that the subject technology may include other configurations whereby a support reference for the compression springs is caused to move so as to increase or reduce tension in the compression springs, and consequently increase or reduce the force exerted by the pumping elements 68 on the fluid tubing loaded within the infusion pump 10.

FIG. 4A depicts a first example of an infusion pump that includes spring-loaded pumping elements with an adjustable preload. One or more linkage arms 122 are rotatably connected between a (movable) support reference 116 and a housing 114 of the pumping mechanism 70. In the depicted example, the linkage arms 122 are in a parallelogram configuration. The actuator device 118 causes the movable support reference 116 to move in a (lateral) path (124) that is perpendicular to a motion of the pumping elements 68 while being constrained by the one or more linkage arms 122. In this regard, the movable support reference 116 moves away from the housing 114 in a direction (120) away from the fluid tubing of pumping segment 66 (and the portion of the pumping elements connected to compression springs 112), thereby reducing the preload/tension in compression springs 112. A spring element 125 may be included on an arm 127 of the actuator device that is responsible for moving the support reference 116, for example, along the lateral path (124) between a lateral end of the support reference 116 and a portion of the housing 114 (e.g., an appendage). The spring element 125 may apply a force to return the movable support reference 116 back to a default position (e.g., move in the opposite direction) when the activator device 118 is deactivated.

FIG. 4B depicts a second example of an infusion pump that includes spring-loaded pumping elements with an adjustable preload. One or more flexure elements 126 are connected to both the (movable) support reference 116 and the housing 114 of the pumping mechanism 70. In the depicted example, the flexure elements 126 are made of a flexible steel or metal alloy. The actuator pulls the movable support reference 116 in a (lateral) direction (124) that is perpendicular to a motion (120) of the pumping elements 68, causing each of the flexure elements 126 to flex onto itself while flexing or expanding in a direction away from the tubing (and the portion of the pumping elements connected to compression springs 112), thereby reducing the preload/tension in compression springs 112. Implementation of the flexure elements 126 may provide a smaller form factor and smaller range of movement than the example mechanisms of FIGS. 4A and 4C. The flexure elements 126 have a benefit of flexing onto themselves to expand outwardly in the direction of the pumping elements 68 when pulled laterally (124) by a mechanical action, while returning to a default state (e.g., move right) when released (e.g., not requiring a spring element 125).

FIG. 4C depicts a third example of an infusion pump that includes spring-loaded pumping elements with an adjustable preload. In this example, the movable support reference 116 is configured to move between the housing 114 and a fixed support 128. The pumping mechanism 70 includes one or more compression springs 130 positioned between the movable support reference 116 and the fixed support 128 such that the movable support reference is biased toward the pumping elements 68 (e.g., occluders 72, 76) and contributes to the compression force applied to the fluid tubing via the spring loaded pumping elements 68. When activated, the actuator 118 causes (e.g., via arm 127) the movable support reference 116 to move away from the housing 114 (and the portion of the pumping elements connected to compression springs 112), reducing the compression force applied to the fluid tubing. The movable support reference 116 moves toward the fixed support thereby compressing the one or more compression springs 130 positioned between the movable support reference 116 and the fixed support 128, while relieving the preload/tension in compression springs 112 and the compression force applied against the tubing by the pumping elements 68. In some implementations, the actuator 118 may be configured to push the movable support reference 116 away in one direction whereas, when released, the support reference 116 is pushed back to a default state by way of the tension created in the compression springs 130.

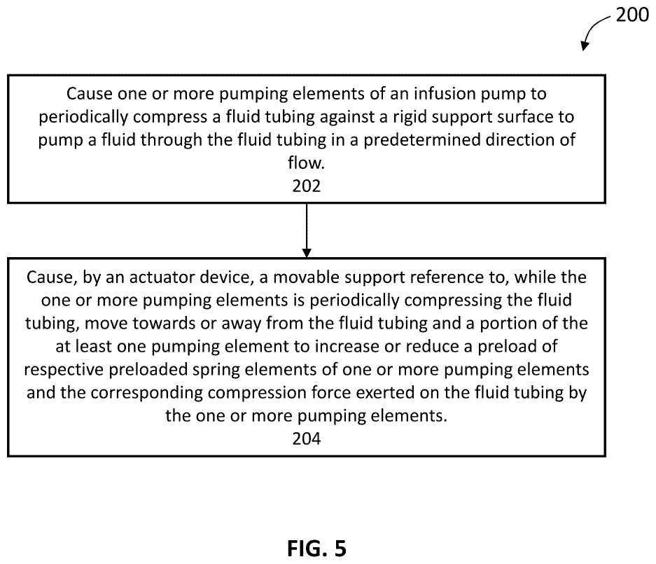

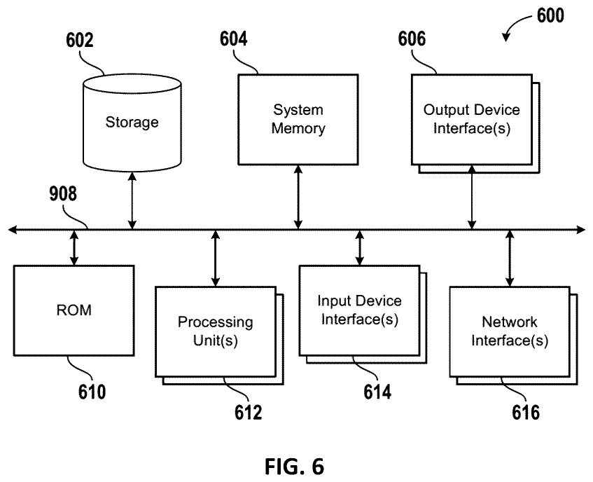

FIG. 5 depicts an example process 200 for operating an infusion pump that includes spring-loaded pumping elements with an adjustable preload for preserving elasticity of a fluid tubing loaded within the pump, according to aspects of the subject technology. For explanatory purposes, the various blocks of example process 200 are described herein with reference to FIGS. 1 through 4, and the components and/or processes described herein. The one or more of the blocks of process 200 may be implemented, for example, by the infusion pump 10 described herein. For example, one or more of the blocks may be implemented based on electromechanical and/or computer control by the pump's electronic subsystems (see, e.g., FIG. 6). For explanatory purposes, the blocks of example process 200 are described as occurring in serial, or linearly. However, the blocks of example process 200 may occur in parallel. In addition, the blocks of example process 200 need not be performed in the order shown and/or one or more of the blocks of example process 200 need not be performed.

In the depicted example, a controller in connection with a pumping mechanism of an infusion pump 10 causes one or more pumping elements to periodically compress a fluid tubing (e.g., pumping segment 66) against a rigid support surface to pump a fluid through the fluid tubing in a predetermined direction of flow (202). According to various implementations, as shown in FIGS. 4A-4C, each pumping element is biased by a respective preloaded spring element 112 positioned between a portion of the pumping element 72,74, 76, 78 and a movable support reference 116 to exert a corresponding compression force on the fluid tubing.

An actuator device 118, associated with the infusion pump 10 and/or pumping mechanism, causes the movable support reference to, while the one or more pumping elements is periodically compressing the fluid tubing, move towards or away from the fluid tubing and the portion of the at least one pumping element so as to increase or reduce the preload of the respective preloaded spring elements, and the corresponding compression force exerted on the fluid tubing by the one or more pumping elements (204).

In some implementations, the movable support reference is caused to move based on a measured pressure. For example, a pressure of the fluid within the tubing may be measured by a pressure sensor 80. The movable support reference may be, for example, caused to move away from the fluid tubing and the portion of the at least one pumping element due to a decrease in the measured pressure and to move towards the fluid tubing and the portion of the at least one pumping element due to an increase in the measured pressure, thereby adjusting the preload in the preloaded spring element(s) 112.

In some implementations, the measured pressure is determined (e.g., by the controller) to have decreased to a first pressure threshold for a first predetermined period of time. Accordingly, the movable support reference may be moved away from the fluid tubing and the portion of the at least one pumping element to reduce the preload so that the corresponding compression force is reduced to a first predetermined compression force responsive to the measured pressure satisfying the first pressure threshold for the first predetermined period of time.

According to various implementations, an algorithm is operational on the controller to control the movement of the support reference 116 based on several factors. For example, the algorithm may include windowing function monitors pressure. If the pressure increases to above an upward threshold then the support reference 116 may be moved to increase the preload on the compression springs 112 so that the tubing occludes to a satisfactory degree (e.g., complete occlusion). If the pressure drops such that the system can occlude adequately with lower preload on springs and stay there for a sufficient period of time then the algorithm may back off the preload by a certain amount. After the adjustment, the window can shift and establish a new threshold and if the (new) lower threshold is again met then the algorithm can back off the preload again.

After the compression force exerted on the fluid tubing by the one or more pumping elements is reduced a first time (e.g., to a first predetermined compression force), the algorithm may set a second threshold for a further reduction. When the algorithm determines that the measured pressure satisfies the second pressure threshold for a second predetermined period of time (e.g., lower than the first pressure threshold), the algorithm may then cause the movable support reference 116 to further move away to further reduce the preload so that the corresponding compression force is further reduced to a second predetermined compression force. If the pressure increases back to the second pressure threshold, the algorithm immediately moves the movable support reference back towards the fluid tubing and the portion of the at least one pumping element to increase the preload so that the corresponding compression force is restored to the first predetermined compression force.

For example, at the start of infusion start, the springs 112 may be preloaded for maximum force. Over time, the support reference 116 is moved to step down the preload to the lowest safe level for the pressure measured in the fluid tubing (e.g., by the sensor 80). The time to stay below the lowest threshold may be predetermined. For example, the algorithm may start with a preload to occlude the fluid tubing having 1200 mm Hg. If the pressure stays below 600 mm Hg for 15 seconds then the algorithm may back off the preload/occlusion force to occlude 750 mm Hg. Then, if the pressure stays below 300 mm Hg for 15-20 seconds then the algorithm can back off the preload/occlusion force so that there is sufficient force to occlude 450 mm Hg. If the pressure is detected above 300 mm Hg then the algorithm may immediately increase preload to occlude 750 mm Hg. In this regard, there is no delay on reapplying occlusion force but there is a delay on reducing the occlusion force.

The algorithm defaults a preload on compression springs 112 to a maximum force, and backs off as pressure reduces. At high flow rates, pressure increases may occur quicker. At lower flow rates it could be hours to get to 500 mm Hg from 100 mm Hg. However, the a quicker change can occur when a patient sits up or lays down. In this regard, the algorithm may detect the threshold being met and adjust the preload accordingly.

In some implementations, one or more of the pressure thresholds for preload adjustments are based on information available to the algorithm that pertains to a given therapy. In this regard, algorithm may determine one or more parameters based on a characterization of the fluid tubing, a characterization of a patient, a therapy, or a fluid type of the fluid within the fluid tubing, and set the (e.g., first or second) pressure thresholds or the (first or second) predetermined period of time based on the determined parameters. The pump 10 or associated system may store a lookup table that maps pressures to force, and which may be indexed by the type of a given IV set. For example, an infusion set A may be mapped to a 600 mm Hg pressure threshold, while an infusion set B may be mapped to an 800 mm Hg threshold. Each pressure threshold may be further mapped to a particular occlusion force or preload force, and the movable support reference is adjusted accordingly to obtain the occlusion force or preload force.

Many of the above-described devices, systems and methods, may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMS, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for operating an infusion pump that includes spring-loaded pumping elements with an adjustable preload for preserving elasticity of a fluid tubing loaded within the pump, according to aspects of the subject technology. Electronic system 600 may be representative of a control unit and/or computing device for execution of software associated with one or more components and processes provided by FIGS. 1 through 5, including but not limited to infusion pump 10 (e.g., a processing system of controller 14 or within infusion pump 10). Electronic system 600 may be representative of a device used in connection or combination with the disclosure regarding FIGS. 1 through 5. In this regard, electronic system 600 may be a device connected to the infusion pump 10, for example, to activate the occluders and/or pumping fingers, the cam of the pumping mechanism, or to monitor or control same. For example, system 600 may be representative of a personal computer or a mobile device such as a smartphone, tablet computer, laptop, personal digital assistant (PDA), an augmented reality device, a wearable such as a watch or band or glasses, or combination thereof, or other touch screen or television with one or more processors embedded therein or coupled thereto, or any other sort of computer-related electronic device having network connectivity specifically configured to implement one or more of the features described.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves specific instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random-access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, a personal area network (“PAN”), or an Intranet, or a network of networks, such as the Internet. Any or all components of electronic system 600 can be used in conjunction with the subject disclosure.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of specific instructions for performing various operations described herein. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs) specifically configured with one or more of the features described. In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a specifically configured computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of specifically configured devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other (e.g., physically separated) and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An infusion pump, comprising: a pumping mechanism comprising a housing and at least one pumping element configured to periodically compress a fluid tubing filled with a fluid against a rigid support surface when the fluid tubing is received by the infusion pump to pump a fluid through the fluid tubing in a predetermined direction, a movable support reference configured to move with respect to the housing of the pumping mechanism; at least one preloaded spring element positioned between the movable support reference and a portion of the at least one pumping element, causing the at least one pumping element to exert a compression force on the fluid tubing thereby compressing the fluid tubing against the rigid support surface; and an actuator device configured to cause, while the at least one pumping element is periodically compressing the fluid tubing, the movable support reference to move towards or away from the housing and the portion of the at least one pumping element so as to increase or reduce the preload of the at least one preloaded spring element and the corresponding compression force exerted on the fluid tubing by the one or more pumping elements.

Clause 2. The infusion pump of Clause 1, further comprising: at least one pressure sensor configured to monitor a pressure of the fluid within the fluid tubing; and a processor configured to: activate the at least one pumping element to compress the fluid tubing; and determine, using the at least one pressure sensor, while the at least one pumping element is periodically compressing the fluid tubing, that the pressure within the fluid tubing satisfies a first pressure threshold for a predetermined period of time; responsive to the pressure satisfying the first pressure threshold for the predetermined period of time, cause the actuator device to move the movable support reference away from the housing and the portion of the at least one pumping element so as to reduce the preload of the at least one preloaded spring element and the corresponding compression force exerted on the fluid tubing.

Clause 3. The infusion pump of Clause 2, wherein the infusion pump is programmed to deliver the fluid according to a therapy for a patient, wherein the processor is further configured to: determining one or more parameters based on a characterization of the fluid tubing, a characterization of the patient, the therapy, or a fluid type of the fluid within the fluid tubing; and determine the first pressure threshold or the predetermined period of time based on the determined one or more parameters.

Clause 4. The infusion pump of Clause 2, wherein the processor is further configured to: determine, using the at least one pressure sensor, while the at least one pumping element is periodically compressing the fluid tubing, that the pressure within the fluid tubing satisfies a second pressure threshold; responsive to the pressure satisfying the second pressure threshold, causing the actuator device to move the movable support reference to a default position so as to restore a default compression force exerted on the fluid tubing.

Clause 5. The infusion pump of Clause 2, wherein the actuator device comprises a motor, and the motor is activated to move the movable support reference.

Clause 6. The infusion pump of Clause 2, wherein the actuator device comprises a piezoelectric actuator.

Clause 7. The infusion pump of Clause 2, wherein the actuator device comprises a hydraulic actuator.

Clause 8. The infusion pump of Clause 1, further comprising: one or more linkage arms rotatably connected to both the movable support reference and a housing of the pumping mechanism, wherein the actuator device being caused to move the movable support reference away from the portion of the at least one pumping element comprises: the movable support reference being caused to move in a first direction perpendicular to a motion of the at least one pumping element while being constrained by the one or more linkage arms, which cause the movable support reference to move away from the housing and the portion of the at least one pumping element when the movable support reference is moved in the first direction, wherein the one or more linkage arms cause the movable support reference to move towards the portion of the at least one pumping element when the movable support reference is moved in a second direction opposite the first direction.

Clause 9. The infusion pump of Clause 1, further comprising: one or more flexure elements connected to both the movable support reference and a housing of the pumping mechanism, wherein the actuator device causing the movable support reference to move away from the portion of the at least one pumping element comprises the actuator device pulling the movable support reference in a first direction to cause each of the one or more flexure elements to flex onto itself and expand in a direction that is perpendicular to the first direction thereby causing the movable support reference to move away from the housing and the portion of the at least one pumping element.

Clause 10. The infusion pump of Clause 9, wherein, when the actuator device releasing the movable support reference causes the movable support reference to automatically move in a second direction opposite the first direction, the one or more flexure elements returning to a default state thereby moving the movable support reference towards the housing and the portion of the at least one pumping element.

Clause 11. The infusion pump of Clause 1, wherein the movable support reference is configured to move between the housing and a fixed support, and wherein the infusion pump further comprises: one or more compression springs positioned between the movable support reference and the fixed support such that the movable support reference is biased by the one or more compression springs toward the at least one pumping element to preload the at least one preloaded spring element, wherein the actuator device being caused to move the movable support reference away from the housing and the portion of the at least one pumping element comprises the movable support reference being moved toward the fixed support to reduce the preload of the at least one preloaded spring element while increasing a preload of the one or more compression springs positioned between the movable support reference and the fixed support.

Clause 12. The infusion pump of Clause 1, wherein the rigid support surface is supported by one or more support springs, wherein a force provided by the one or more support springs resists and yields to the compression force on the fluid tubing exerted by the at least one pumping element to facilitate an even distribution of pressure along the tubing.

Clause 13. A method comprising: causing one or more pumping elements of an infusion pumping mechanism to periodically compress a fluid tubing against a rigid support surface to pump a fluid through the fluid tubing in a predetermined direction of flow, each pumping element being biased by a respective preloaded spring element positioned between a portion of the pumping element and a movable support reference to exert a corresponding compression force on the fluid tubing; and causing, by an actuator device, the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.

Clause 14. The method of Clause 13, further comprising: measuring a pressure of the fluid within the fluid tubing; and causing the movable support reference to move based on the measured pressure, wherein the movable support reference is caused to move away from the fluid tubing and the portion of the at least one pumping element due to a decrease in the measured pressure and to move towards the fluid tubing and the portion of the at least one pumping element due to an increase in the measured pressure.

Clause 15. The method of Clause 14, further comprising: determining that the measured pressure decreased to a first pressure threshold for a first predetermined period of time, wherein the movable support reference is moved away from the fluid tubing and the portion of the at least one pumping element to reduce the preload so that the corresponding compression force is reduced to a first predetermined compression force responsive to the measured pressure satisfying the first pressure threshold for the first predetermined period of time.

Clause 16. The method of Clause 15, further comprising, after the compression force exerted on the fluid tubing by the one or more pumping elements is reduced to the first predetermined compression force: determining that the measured pressure satisfies a second pressure threshold for a second predetermined period of time, the second pressure threshold being lower than the first pressure threshold; and responsive to the measured pressure satisfying the second pressure threshold for the second predetermined period of time, causing the movable support reference to further move away to further reduce the preload so that the corresponding compression force is further reduced to a second predetermined compression force; and causing the movable support reference to immediately move back towards the fluid tubing and the portion of the at least one pumping element to increase the preload so that the corresponding compression force is restored to the first predetermined compression force.

Clause 17. The method of Clause 15, further comprising, after the compression force exerted on the fluid tubing by the one or more pumping elements is reduced to the first predetermined compression force: determining one or more parameters based on a characterization of the fluid tubing, a characterization of a patient, a therapy, or a fluid type of the fluid within the fluid tubing; and determining the first pressure threshold or the first predetermined period of time based on the determined one or more parameters.

Clause 18. The method of Clause 13, further comprising, responsive to a predetermined condition: causing the movable support reference to automatically move to a default position so as to restore a default compression force exerted on the fluid tubing.

Clause 19. The method of Clause 13, wherein causing the movable support reference to move comprises: the actuator device laterally moving the movable support reference in a direction that is perpendicular to the one or more pumping elements, wherein the movable support reference is linked to a housing of the infusion pumping mechanism by one or more linkage arms or one or more flexure elements that cause the movable support reference to move away from the fluid tubing when the movable support reference is moved in a first lateral direction and to move towards the fluid tubing when the movable support reference is moved in a second lateral direction, opposite the first lateral direction.

Clause 20. A non-transitory machine readable medium storing instructions thereon that, when executed by a machine, cause the machine to perform operations comprising: causing one or more pumping elements of an infusion pumping mechanism to periodically compress a fluid tubing against a rigid support surface to pump a fluid through the fluid tubing in a predetermined direction of flow, each pumping element being biased by a respective preloaded spring element positioned between a portion of the pumping element and a movable support reference to exert a corresponding compression force on the fluid tubing; and causing, by an actuator device, the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.

### Further Consideration:

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. An infusion pump, comprising:
a pumping mechanism comprising a housing and at least one pumping element configured to periodically compress a fluid tubing filled with a fluid against a rigid support surface when the fluid tubing is received by the infusion pump to pump a fluid through the fluid tubing in a predetermined direction,
a movable support reference configured to move with respect to the housing of the pumping mechanism;
at least one preloaded spring element positioned between the movable support reference and a portion of the at least one pumping element, causing the at least one pumping element to exert a compression force on the fluid tubing thereby compressing the fluid tubing against the rigid support surface; and
an actuator device configured to cause, while the at least one pumping element is periodically compressing the fluid tubing, the movable support reference to move towards or away from the housing and the portion of the at least one pumping element so as to increase or reduce the preload of the at least one preloaded spring element and the corresponding compression force exerted on the fluid tubing by the one or more pumping elements.

2. The infusion pump of claim 1, further comprising:
at least one pressure sensor configured to monitor a pressure of the fluid within the fluid tubing; and
a processor configured to:
activate the at least one pumping element to compress the fluid tubing; and
determine, using the at least one pressure sensor, while the at least one pumping element is periodically compressing the fluid tubing, that the pressure within the fluid tubing satisfies a first pressure threshold for a predetermined period of time;
responsive to the pressure satisfying the first pressure threshold for the predetermined period of time, cause the actuator device to move the movable support reference away from the housing and the portion of the at least one pumping element so as to reduce the preload of the at least one preloaded spring element and the corresponding compression force exerted on the fluid tubing.

3. The infusion pump of claim 2, wherein the infusion pump is programmed to deliver the fluid according to a therapy for a patient, wherein the processor is further configured to:
determining one or more parameters based on a characterization of the fluid tubing, a characterization of the patient, the therapy, or a fluid type of the fluid within the fluid tubing; and
determine the first pressure threshold or the predetermined period of time based on the determined one or more parameters.

4. The infusion pump of claim 2, wherein the processor is further configured to:
determine, using the at least one pressure sensor, while the at least one pumping element is periodically compressing the fluid tubing, that the pressure within the fluid tubing satisfies a second pressure threshold;
responsive to the pressure satisfying the second pressure threshold, causing the actuator device to move the movable support reference to a default position so as to restore a default compression force exerted on the fluid tubing.

5. The infusion pump of claim 2, wherein the actuator device comprises a motor, and the motor is activated to move the movable support reference.

6. The infusion pump of claim 2, wherein the actuator device comprises a piezoelectric actuator.

7. The infusion pump of claim 2, wherein the actuator device comprises a hydraulic actuator.

8. The infusion pump of claim 1, further comprising:
one or more linkage arms rotatably connected to both the movable support reference and a housing of the pumping mechanism,
wherein the actuator device being caused to move the movable support reference away from the portion of the at least one pumping element comprises: the movable support reference being caused to move in a first direction perpendicular to a motion of the at least one pumping element while being constrained by the one or more linkage arms, which cause the movable support reference to move away from the housing and the portion of the at least one pumping element when the movable support reference is moved in the first direction,
wherein the one or more linkage arms cause the movable support reference to move towards the portion of the at least one pumping element when the movable support reference is moved in a second direction opposite the first direction.

9. The infusion pump of claim 1, further comprising:
one or more flexure elements connected to both the movable support reference and a housing of the pumping mechanism,
wherein the actuator device causing the movable support reference to move away from the portion of the at least one pumping element comprises the actuator device pulling the movable support reference in a first direction to cause each of the one or more flexure elements to flex onto itself and expand in a direction that is perpendicular to the first direction thereby causing the movable support reference to move away from the housing and the portion of the at least one pumping element.

10. The infusion pump of claim 9, wherein, when the actuator device releasing the movable support reference causes the movable support reference to automatically move in a second direction opposite the first direction, the one or more flexure elements returning to a default state thereby moving the movable support reference towards the housing and the portion of the at least one pumping element.

11. The infusion pump of claim 1, wherein the movable support reference is configured to move between the housing and a fixed support, and wherein the infusion pump further comprises:
one or more compression springs positioned between the movable support reference and the fixed support such that the movable support reference is biased by the one or more compression springs toward the at least one pumping element to preload the at least one preloaded spring element,
wherein the actuator device being caused to move the movable support reference away from the housing and the portion of the at least one pumping element comprises the movable support reference being moved toward the fixed support to reduce the preload of the at least one preloaded spring element while increasing a preload of the one or more compression springs positioned between the movable support reference and the fixed support.

12. The infusion pump of claim 1, wherein the rigid support surface is supported by one or more support springs, wherein a force provided by the one or more support springs resists and yields to the compression force on the fluid tubing exerted by the at least one pumping element to facilitate an even distribution of pressure along the tubing.

13. A method comprising:
causing one or more pumping elements of an infusion pumping mechanism to periodically compress a fluid tubing against a rigid support surface to pump a fluid through the fluid tubing in a predetermined direction of flow, each pumping element being biased by a respective preloaded spring element positioned between a portion of the pumping element and a movable support reference to exert a corresponding compression force on the fluid tubing; and
causing, by an actuator device, the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.

14. The method of claim 13, further comprising:
measuring a pressure of the fluid within the fluid tubing; and
causing the movable support reference to move based on the measured pressure,
wherein the movable support reference is caused to move away from the fluid tubing and the portion of the at least one pumping element due to a decrease in the measured pressure and to move towards the fluid tubing and the portion of the at least one pumping element due to an increase in the measured pressure.

15. The method of claim 14, further comprising:
determining that the measured pressure decreased to a first pressure threshold for a first predetermined period of time,
wherein the movable support reference is moved away from the fluid tubing and the portion of the at least one pumping element to reduce the preload so that the corresponding compression force is reduced to a first predetermined compression force responsive to the measured pressure satisfying the first pressure threshold for the first predetermined period of time.

16. The method of claim 15, further comprising, after the compression force exerted on the fluid tubing by the one or more pumping elements is reduced to the first predetermined compression force:
determining that the measured pressure satisfies a second pressure threshold for a second predetermined period of time, the second pressure threshold being lower than the first pressure threshold; and
responsive to the measured pressure satisfying the second pressure threshold for the second predetermined period of time, causing the movable support reference to further move away to further reduce the preload so that the corresponding compression force is further reduced to a second predetermined compression force; and
causing the movable support reference to immediately move back towards the fluid tubing and the portion of the at least one pumping element to increase the preload so that the corresponding compression force is restored to the first predetermined compression force.

17. The method of claim 15, further comprising, after the compression force exerted on the fluid tubing by the one or more pumping elements is reduced to the first predetermined compression force:
determining one or more parameters based on a characterization of the fluid tubing, a characterization of a patient, a therapy, or a fluid type of the fluid within the fluid tubing; and
determining the first pressure threshold or the first predetermined period of time based on the determined one or more parameters.

18. The method of claim 13, further comprising, responsive to a predetermined condition:
causing the movable support reference to automatically move to a default position so as to restore a default compression force exerted on the fluid tubing.

19. The method of claim 13, wherein causing the movable support reference to move comprises:
the actuator device laterally moving the movable support reference in a direction that is perpendicular to the one or more pumping elements, wherein the movable support reference is linked to a housing of the infusion pumping mechanism by one or more linkage arms or one or more flexure elements that cause the movable support reference to move away from the fluid tubing when the movable support reference is moved in a first lateral direction and to move towards the fluid tubing when the movable support reference is moved in a second lateral direction, opposite the first lateral direction.

20. A non-transitory machine readable medium storing instructions thereon that, when executed by a machine, cause the machine to perform operations comprising:
causing one or more pumping elements of an infusion pumping mechanism to periodically compress a fluid tubing against a rigid support surface to pump a fluid through the fluid tubing in a predetermined direction of flow, each pumping element being biased by a respective preloaded spring element positioned between a portion of the pumping element and a movable support reference to exert a corresponding compression force on the fluid tubing; and
causing, by an actuator device, the movable support reference to, while the one or more pumping elements is periodically compressing the fluid tubing, move towards or away from the fluid tubing and the portion of the at least one pumping element so as to increase or reduce the preload of the respective preloaded spring elements and the corresponding compression force exerted on the fluid tubing by the one or more pumping elements.
